(19) United States
(12) Patent Application Publication
(10) Pub. No.: US 2014/0120623 A1
(43) Pub. Date: May 1, 2014

(54) Method for Enhancing Cell Growth of Microalgae

(71) Applicant: Institute of Nuclear Energy Research, Atomic Energy Council, Executive Yuan, Taoyuan County (TW)

(72) Inventors: Jia-Baau Wang, Taipei City (TW); Sheng-Hsin Chou, Taoyuan County (TW); Te-Jin Chow, Kaohsiung City (TW); Tse-Min Lee, Kaohsiung City (TW); Hsiang-Yen Su, Chiayi County (TW); Hsiang-Hsu Chou, Taichung City (TW); Yuan-Ting Hsu, Kaohsiung City (TW); Yu-Rong Pan, Pingtung County (TW)

(73) Assignee: Institute of Nuclear Energy Research, Atomic Energy Council, Executive Yuan, Taoyuan County (TW)

(21) Appl. No.: 13/868,639

(22) Filed: Apr. 23, 2013

(30) Foreign Application Priority Data
Oct. 31, 2012 (TW) 101140254

(51) Int. Cl. C12N 15/82

(52) U.S. Cl. CPC C12N 15/8243
USPC 435/476

(57) ABSTRACT

Microalgae are potential energy resources for production of biofuels, such as biodiesel, ethanol, and butanol. A method for enhancing cell growth of microalgae enhances transgenic expression of a bicarbonate transporter (HCO₃⁻ transporter) in microalgae and thereby obtains a genetically modified microalgae capable of enhanced inorganic carbon fixation, efficient photosynthesis, and expeditious cell growth. The genetically modified microalgae are fit for use in biofuel production.

### CROSS-REFERENCE TO RELATED APPLICATION

This non-provisional application claims priority under 35 U.S.C. §119(a) on Patent Application No(s).101140254 filed in Taiwan, R.O.C. on Oct. 31, 2012, the entire contents of which are hereby incorporated by reference.

### FIELD OF THE INVENTION

The present invention relates to methods for enhancing cell growth of microalgae, and more particularly, to a method for enhancing transgenic expression of a bicarbonate transporter (HCO₃⁻ transporter) in microalgae by gene transfer to thereby enhance inorganic carbon fixation in microalgae, enhance photosynthesis and cell growth of microalgae, and apply a genetically modified microalgae to production of biofuels.

### BACKGROUND OF THE INVENTION

With global fossil fuel resources dwindling, development of renewable energy resources is all the rage today. An appealing alternative energy source, bioethanol is produced from various forms of biomass and by biotransfer. At present, bioethanol is produced mostly from terrestrial plant cellulose, such as corn cellulose, sugarcane cellulose, and wood cellulose. However, high production costs and shortage of raw materials are among the major limiting factors in the mass production of bioethanol.

Algae abound in waters. Hence, both unauthorized logging and required agricultural land can be reduced, if high-efficiency low-cost microalgae are developed to thereby enable mass production of cellulose and sugar—raw materials of bioethanol.

As a source of bioenergy, an alga has advantages as follows: A. it exhibits high photon conversion efficiency per hectare of biomass; B. it grows throughout the year and thus serves as a reliable year-round energy supplier; C. it feeds on waste water and seawater, thereby recycling resources and reducing pollution; D. it takes in carbon dioxide (CO₂) efficiently and thereby reduces the greenhouse effect; E. it produces bioenergy in a highly biodegradable manner without causing toxicity and hazards; and F. it features high biodiversity in terms of species.

The first-generation vegetation-based bioenergy production resorts mostly to crops at the expense of the supply of human foods and livestock fodder. However, this is not true to algae, which outgrow crops even in adverse growth conditions. Mass production of ethanol will be feasible, provided that microalgae are used as a bioenergy source, for example. The cultivation area required for the microalgae equals just 3.5% that of corn; hence, using microalgae as a bioenergy source is effective in reducing the required agricultural land and unauthorized logging.

As mentioned earlier, the answer to the question as to whether microalgae can be good raw materials for producing biofuels, such as ethanol, biodiesel, and butanol, is the affirmative. The next question for microalgae is how microalgae are cultivated on a large scale, efficiently, and in a high-yield manner.

### SUMMARY OF THE INVENTION

In view of the aforesaid drawbacks of the prior art, it is an objective of the present invention to provide a method for enhancing cell growth of microalgae, so as to enhance photosynthesis efficiency of microalgae, speed up the growth of microalgae, and increase the biomass of microalgae.

In order to achieve the above and other objectives, the present invention provides a method for enhancing cell growth of microalgae by modifying the microalgae through gene transfer. The method is characterized in that transgenic expression of a bicarbonate transporter (HCO₃⁻ transporter) in microalgae is enhanced.

The method is characterized in that the bicarbonate transporter has a DNA sequence known as SEQ ID NO: 1 and is cloned from the ictB gene of microalgae (Synechococcus elongatus PCC7942). The Synechococcus elongatus PCC7942 is purchased from the Pasteur Culture Collection of Cyanobacteria, France.

The method is characterized in that the bicarbonate transporter has a DNA sequence known as SEQ ID NO: 2 and is cloned from the BicA gene of microalgae (Synechococcus PCC7002). The Synechococcus PCC7002 is purchased from the Pasteur Culture Collection of Cyanobacteria, France.

The method is characterized in that the vector for transgenic expression of a bicarbonate transporter in microalgae is upgraded to a transgenic vector pAM1573 (wherein the pAM1573 vector is put forth by Susan S. Golden, distinguished Professor, Section of Molecular Biology, UCSD.

The method is characterized in that the microalgae is Synechococcus, Thermosynechococcus, Cyanothece, Anabaena, Chlorella, or Chlamydomonas reinhardtii.

Among the major limiting factors in photosynthesis is that carbon dioxide accounts for only 0.03% of the chemical composition of the Earth's atmosphere, and that aquatic environments where aquatic plants live usually have low carbon dioxide concentration (though bicarbonates account for about 99% of aquatic carbon content). Hence, the objective of the present invention is to increase by gene transfer the genes (ictB or BicA) responsible for a bicarbonate transporter (HCO₃⁻ transporter) for delivering bicarbonates in microalgae to thereby increase accumulation of bicarbonates in microalgae, turn the bicarbonates into carbon dioxide with carbonic anhydrase in microalgae, produce carbonhydrates from carbon dioxide by means of ribulose-1, 5-bisphosphatecarboxylase, to thereby speed up photosynthesis and enhance production yield.

The genetically modified microalgae produced by the method of the present invention have the following advantages:
1. The genetically modified microalgae thus produced incur low cultivation costs. It is because microalgae are photoautotrophs which carry out photosynthesis, using waste water not fit to be used by human beings and crops. Furthermore, airborne carbon dioxide fixation is achieved as a result of the photosynthesis carried out by microalgae, thereby reducing air pollution.
2. Unlike the conventional bioenergy production process that requires extracting saccharides from crops, the method of the present invention dispenses with a processing process which might otherwise be required for the conventional bioenergy production process that uses the other woody plants or herbaceous plants, not to mention that the method of the present invention is further characterized in that saccharides and cellulose secreted by microalgae can be continuously collected without affecting the growth of microalgae. Some microalgae fix atmospheric nitrogen and thus require no nitrogen fertilizer.

### BRIEF DESCRIPTION OF THE DRAWINGS

Objectives, features, and advantages of the present invention are hereunder illustrated with specific embodiments in conjunction with the accompanying drawings, in which:

FIG. 1 is a schematic view of a portion of an PrbcL-ictB transgenic vector;

FIG. 2 shows graphs of the growth rate of a transgenic strain (PrbcL-ictB) and a control strain against the concentration of airborne carbon dioxide;

FIG. 3 is a bar chart of the photosynthesis rate of the transgenic strain (PrbcL-ictB) and a control strain against the concentration of airborne carbon dioxide;

FIG. 4 is a partial schematic view of an PrbcL-BicA transgenic vector;

FIG. 5 is a bar chart of the biomass yield of a transgenic strain (PrbcL-BicA) and a control strain which grow at 2% airborne carbon dioxide;

FIG. 6 is a bar chart of the biomass yield of the transgenic strain (PrbcL-BicA) and a control strain which grow in 50 mM of NaHCO₃ solution; and

FIG. 7 is a bar chart of the photosynthesis rate of the transgenic strain (PrbcL-BicA) and a control strain which grow in 50 mM of NaHCO₃ solution.

### DETAILED DESCRIPTION OF THE PREFERRED EMBODIMENTS

### Embodiment 1

### Synechococcus elongatus PCC7942 Bicarbonate Transporter ictB Transgenic Strain Preparation

1. Cloning of ictB Gene

The bicarbonate transporter ictB gene is cloned from Synechococcus elongatus PCC7942, and the ictB gene primer pair (shown in Table 1) is designed. A chromosome gene (chromosomal DNA) of Synechococcus elongatus PCC7942 functions as a template. A polymerase chain reaction (PCR) is carried out by means of the ictB gene primer pair. The PCR reagent solution contains 1× PCR buffer solution, 0.4 mM of dNTP, 2 mM of MgCl₂, 1 unit of Takara ex Taq DNA polymerase, and 0.5 μM of primer (ictB-f, ictB-r), has a total volume of 50 μL, and reacts at 95° C. for 3 minutes; 32 cycles: at 95° C. for 1 minute, at 55° C. for 1 minute, at 72° C. for 2 minutes; and eventually the polymerase chain reaction process is extended at 72° C. for 10 minutes, and at 4° C. continuously, so as for the polymerase chain reaction to increase the ictB gene segment and allow the increased ictB gene segment to be bound to pGEM-T (Promega Corporation, Madison, Wis.) plasmid by means of T4 DNA ligase to thereby obtain the ictB gene-containing pGEM-T-ictB plasmid.

TABLE 1
ictB primer pair

| primer | 5′ → 3′ |
|---|---|
| ictB-for | AAGAATTCGGATCCATGACTGTCTGG |
| ictB-rev | AGGAATTCGGTACCCTACATTTTTTCGT |

### 2. PrbcL-ictB Gene Transfer Vector Construction

The transgenic vector pAM1573 is treated with restriction enzyme EcoRV, and then treated with Alkaline Phosphatase (New England Biolabs, USA), to prevent DNA self-ligation.

The ictB gene segment is cleaved off from the pGEM-T-ictB plasmid by means of restriction enzyme EcoRI (New England Biolabs, USA). Then, the two ends of the DNA are trimmed with Klenow enzyme (New England Biolabs, USA). Afterward, by ligation, the gene segment in its entirety is inserted into the EcoRV cleavage site of the transgenic vector pAM1573 of Synechococcus elongatus PCC7942. Finally, the ligated DNA undergoes heat shock transformation to enter E. coli DH5α, thereby obtaining the ictB gene transfer vector pAM1573-ictB of Synechococcus elongatus PCC7942.

The gene transfer vector pAM1573-ictB of Synechococcus elongatus PCC7942 is treated with restriction enzyme SmaI and Alkaline Phosphatase (New England Biolabs, USA) to prevent DNA self-ligation.

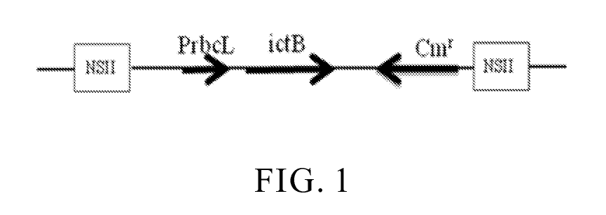

Then, Synechococcus elongatus PCC7942 rbcL promoter gene segment is cleaved off from pYT&A-rbcL plasmid (Te-Jin Chow, Fooyin University, Taiwan) by means of restriction enzyme SmaI (New England Biolabs, USA). Afterward, by ligation, the gene segment in its entirety is inserted into the SmaI cleavage site of transgenic vector pAM1573-ictB of Synechococcus elongatus PCC7942. Then, the ligated DNA undergoes heat shock transformation to enter E. coli DH5α, thereby obtaining ictB gene transfer vector PrbcL-ictB of Synechococcus elongatus PCC7942. Referring to FIG. 1, there is shown a schematic view of a portion of the PrbcL-ictB transgenic vector.

### Transformation of Microalgae

With a centrifugal separation process, 10 mL of Synechococcus sp. PCC7942 cell is collected. Then, remove the culture solution, and add 5 mL of 10 mM NaCl solution. Then, the resultant solution is mixed and subjected to the centrifugal separation process again at 3,980 rpm for 10 minutes. Then, remove the supernatant, and add 1 mL of 10 mM EPPS-containing BG-11 liquid culture suspension of algal cells. Then, add 1.5 μg of plasmid DNA PrbcL-BicA and PrbcL-ictB extracted by Mini Plus™ Plasmid DNA Extraction System (VIOGENE-Bio Tek, Taipei, Taiwan). Afterward, put the mixture in a dark oscillation culture medium at 28° C. overnight. Then, on the following day, the mixture is irradiated for six hours before being treated with the centrifugal separation process again at 14,000 rpm for two minutes to collect algal cells. Afterward, add 300 μL of 10 mM EPPS-containing BG-11 liquid culture suspension of algal cells. Then, the mixture is applied to 10 mM EPPS-containing BG-11 solid culture medium, and the mixture is applied to chloramphenicol (7.5 μg mL⁻¹, Sigma, USA)-containing BG-11 solid culture medium. Eventually, both the mixture-coated EPPS-containing BG-11 solid culture medium and the mixture-coated chloramphenicol-containing BG-11 solid culture medium are cultured under irradiation at room temperature until algal colonies begin to grow.

The algal colonies on the solid culture medium are picked out with a sterilized toothpick, put on a chloramphenicol (7.5 μg mL⁻¹)-containing solid culture medium, and cultured under irradiation at room temperature for two weeks. Afterward, well-grown algal strains are moved to a chloramphenicol (7.5 μg mL⁻¹)-containing BG-11 liquid culture medium.

3. Synechococcus elongatus PCC7942 Bicarbonate Transporter ictB Transgenic Strain Preparation

The ictB transgenic vector PrbcL-ictB (rbcL promoter-ictB) undergoes transformation to therefore be transferred to wild-type Synechococcus sp. PCC7942, and then it is treated with antibiotic Chloramphenicol to perform transgenic alga selection. With a centrifugal separation process, 10 mL of Synechococcus sp. PCC7942 cells is collected. Then, remove the culture solution, and add 5 mL of 10 mM NaCl solution. Then, the resultant solution is mixed and subjected to the centrifugal separation process again at 3,980 rpm for 10 minutes. Then, remove the supernatant, and add 1 mL of 10mM EPPS-containing BG-11 liquid culture suspension of algal cells. Then, add 1.5 μg of PrbcL-ictB plasmid DNA. Afterward, put the mixture in a dark oscillation culture medium at 28° C. overnight. Then, on the following day, the mixture is irradiated for six hours before being treated with the centrifugal separation process again at 14,000 rpm for two minutes to collect algal cells. Afterward, add 300 μL of 10 mM EPPS-containing BG-11 liquid culture suspension of algal cells. Then, the resultant mixture is diluted tenfold consecutively. Then, 100 μL of the diluted mixture is applied to 10 mM EPPS-containing BG-11 solid culture medium, and 100 μL of the diluted mixture is applied to Chloramphenicol (7.5 μgmL⁻¹)-containing BG-11 solid culture medium. Afterward, both the diluted mixture-coated mM EPPS-containing BG-11 solid culture medium and the diluted mixture-coated Chloramphenicol-containing BG-11 solid culture medium are cultured by being irradiated at 28° C. until algal colonies begin to grow. The algal colonies on the solid culture medium are picked out with a sterilized toothpick and put on a 10 M EPPS-containing BG-11 solid culture medium and a Spectinomycin (2 μg mL⁻¹)-containing BG-11 solid culture medium and cultured under irradiation. Afterward, well-grown algal strains are moved to a chlorophenicol (7.5 μgmL⁻¹)-containing liquid culture medium and cultured thereon.

The transgenic strains are cultured on an antibiotic-containing culture medium. A substantially complete loop of the transgenic strains or about 1.5 mL of microalgae is scratched and fetched. The microalgae are examined with a colonial polymerase chain reaction to determine whether the microalgae contain an ictB gene. Furthermore, the algal colonies are treated with TE-triton solution (TE, pH 8.0+1% Triton X-100) to achieve cellular suspension, and then the suspension is heated up at 95° C. for 3.5 min before being subjected to chloroform extraction twice. Then, the supernatant is fetched to undergo the polymerase chain reaction with the ictBprimer pair. Eventually, the transgenic microalgae are examined to determine whether they contain any ictB gene. Upon completion of examination, whatever an ictB gene segment-containing transgenic microalga is regarded as a desirable transgenic strain.

4. Effect of CO₂ Concentration on Growth of Synechococcus sp. PCC7942 ictB Transgenic Strain and Photosynthesis Thereof

14 mL of a transgenic strain algal solution which has stayed still and been cultured for about five weeks is added to 500 ml of spectinomycin (2 μg/ml)-containing BG11+EPPS culture solution. The aforesaid mixture is cultured with three filtered gases of different concentration levels of airborne CO₂, namely 0.03% CO₂/air, 2% CO₂/air, and 5% CO₂/air, at a gas passing speed of 32.4 mL/min, at a cultivation temperature of 28° C., with light intensity of 4000 lux, and for a 12 hL/12 hD irradiation cycle. A fresh BG-11 culture solution serves as a blank control. The optical density OD level of the culture solution is measured daily at a specific point in time and at wavelength 750 nm, using an ultraviolet visible spectrophotometer (HITACHI U-2001, Japan). An algal dry weight is calculated according to OD₇₅₀ absorption value, using a graph of algal dry weight against OD₇₅₀ absorption value. Then, a curve of growth of Synechococcus sp. PCC7942 grown at different CO₂ concentration levels is plotted.

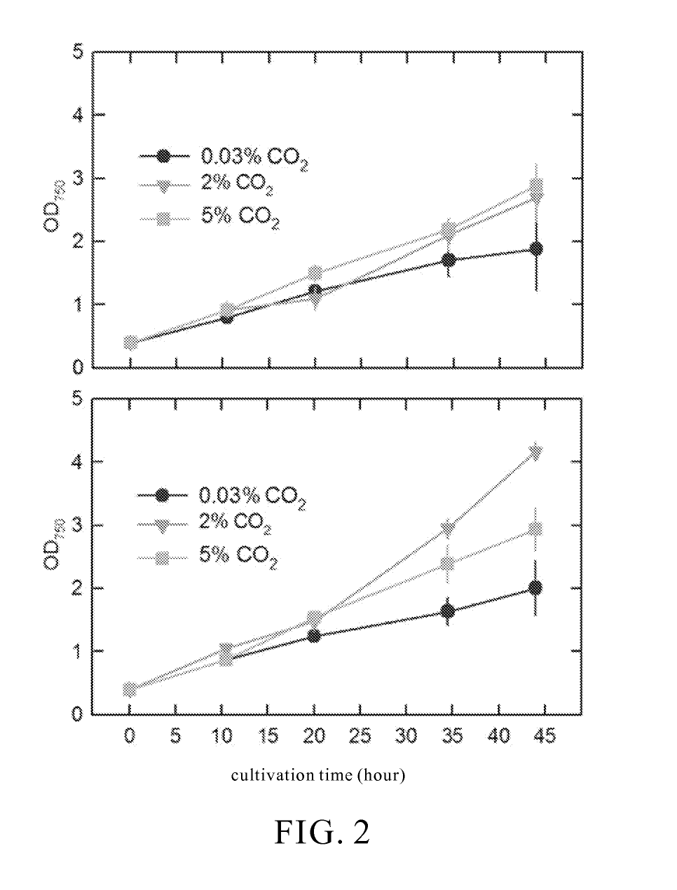

Referring to FIG. 2, under irradiation of 300 E m⁻² s⁻¹, the growth rate (OD₇₅₀) of the control strain and the transgenic strain which are cultured with air (comprising 0.03% CO₂), 2% CO₂, and 5% CO₂ are measured. The result of measurement indicates that discrepancy in the growth rate between the transgenic strain cultured with 2% CO₂ and the transgenic strain cultured with air and 5% CO₂ is unnoticeable until after 20 hours. 45 hours after the cultivation begins, it is obvious that the transgenic strain cultured with 2% CO₂ exhibits a growth rate (OD₇₅₀) of 4.0 approximately which is higher than a growth rate (OD₇₅₀) of 3.0 when cultured with 5% CO₂ and a growth rate (OD₇₅₀) of 2.0 when cultured with air. Hence, the result of measurement proves that the transgenic strain has optimal growth in the 2% CO₂ environment.

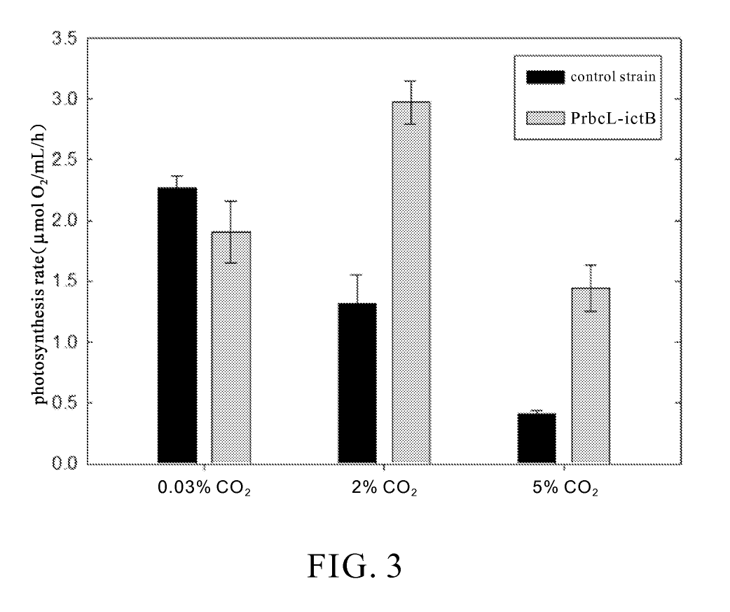

Growth is directly proportional to photosynthesis rate. Hence, the experiment further involves measuring the photosynthesis rate during the fastest growth phase indicated by linearity. Referring to FIG. 3, the result of measurement, which is based on PCC 7942 algal strain-related experimental data, shows that the photosynthesis rate of the algal strains depends on the concentration of CO₂ supplied. Specifically speaking, the photosynthesis rate of the transgenic strain supplied with 2% CO₂ is distinguishable from the photosynthesis rate when supplied with 0.03% CO₂ and 5% CO₂. For example, the photosynthesis rate of the transgenic strain supplied with 2% CO₂ is not only two times the photosynthesis rate of the control strain but also significantly higher than the photosynthesis rate of the transgenic strain supplied with 0.03% CO₂ and 5% CO₂. Hence, the result of measurement proves that the transgenic strain exhibits the highest photosynthesis rate and thus optimal growth in the 2% CO₂ environment.

### Embodiment 2

### Synechococcus elongatus PCC7942 Bicarbonate Transporter BicA Transgenic Strain Preparation

### 1. Cloning of BicA Gene

The bicarbonate transporter BicA gene is cloned from Synechococcus sp. PCC7002, and the BicA gene primer pair (shown in Table 2) is designed. A chromosome gene (chromosomal DNA) of Synechococcus sp. PCC7002 functions as a template. A polymerase chain reaction (PCR) is carried out by means of the BicA gene primer pair. The PCR reagent solution contains 1× PCR buffer solution, 0.4 mM of dNTP, 2 mM of MgCl₂, 1 unit of Takara ex Taq DNA polymerase, and 0.5 μM of primer (BicA-f, BicA-r), has a total volume of 50 μL, and reacts at 95° C. for 3 minutes; 32 cycles: at 95° C. for 1 minute, at 55° C. for 1 minute, at 72° C. for 2 minutes; and eventually the polymerase chain reaction process is extended at 72° C. for 10 minutes, and at 4° C. continuously, so as for the polymerase chain reaction to increase the BicA gene segment and allow the increased BicA gene segment to be bound to yT&A (Yeastern Biotech Co., Ltd.) plasmid by means of T4 DNA ligase to thereby obtain the BicA gene-containing pYT&A-BicA plasmid.

TABLE 1
BicA Primer Pair

| primer | 5′ → 3′ |
|---|---|
| BicA -for | AATTCCCGGGTTTAAGAAGGAGATATACATATGCAGA |
| | TAACCAACAAAATTCACT |
| BicA-rev | AATTCCCGGGTTAACCCATCTCTGAACTGGG |

### 2. PrbcL-BicA Gene Transfer Vector Construction

The rbcL promoter-carrying transgenic vector pAM1573-PrbcL (Te-Jin Chow, Fooyin University, Taiwan) is treated with restriction enzyme EcoRV, and then treated with Alkaline Phosphatase (New England Biolabs, USA), to prevent DNA self-ligation.

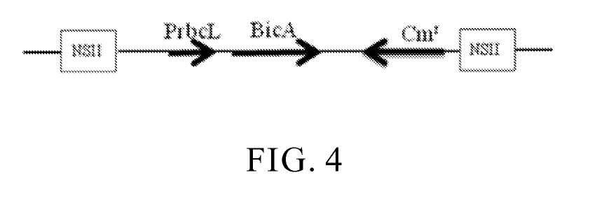

The BicA gene segment is cleaved off from the pYT&A-BicA plasmid by means of restriction enzyme SmaI (New England Biolabs, USA). Then, by ligation, the gene segment in its entirety is inserted into the cleavage site of EcoRV of transgenic vector pAM1573-PrbcL of Synechococcus sp. PCC7942. Afterward, the ligated DNA undergoes heat shock transformation to enter E. coli DH5α, thereby obtaining BicA gene transfer vector PrbcL-BicA of Synechococcus sp. PCC7942. Referring to FIG. 4, there is shown a schematic view of a portion of the PrbcL-BicA transgenic vector.

3. Synechococcus elongatus PCC7942 Bicarbonate Transporter BicA Transgenic Strain Preparation

The BicA transgenic vector PrbcL-BicA (Tac promoter-BicA) undergoes transformation to therefore be transferred to wild-type Synechococcus sp. PCC7942, and then it is treated with antibiotic Chloramphenicol to perform transgenic alga selection. With a centrifugal separation process, 10 mL of Synechococcus sp. PCC7942 cells is collected. Then, remove the culture solution, and add 5 mL of 10 mM NaCl solution. Then, the resultant solution is mixed and subjected to the centrifugal separation process again at 3,980 rpm for 10 minutes. Then, remove the supernatant, and add 1 mL of 10 mM EPPS-containing BG-11 liquid culture suspension of algal cells. Then, add 1.5 μg of PrbcL-BicA plasmid DNA. Afterward, put the mixture in a dark oscillation culture medium at 28° C. overnight. Then, on the following day, the mixture is irradiated for six hours before being treated with the centrifugal separation process again at 14,000 rpm for two minutes to collect algal cells. Afterward, add 300 μL of 10 mM EPPS-containing BG-11 liquid culture suspension of algal cells. Then, the resultant mixture is diluted tenfold consecutively. Then, 100 μL of the diluted mixture is applied to 10 mM EPPS-containing BG-11 solid culture medium, and 100 μL of the diluted mixture is applied to Chloramphenicol (7.5 μgmL⁻¹)-containing BG-11 solid culture medium. Afterward, both the diluted mixture-coated mM EPPS-containing BG-11 solid culture medium and the diluted mixture-coated Chloramphenicol-containing BG-11 solid culture medium are cultured by being irradiated at 28° C. until algal colonies begin to grow. The algal colonies on the solid culture medium are picked out with a sterilized toothpick and put on a 10 mM EPPS-containing BG-11 solid culture medium and a Spectinomycin (2 μg mL⁻¹)-containing BG-11 solid culture medium and cultured under irradiation. Afterward, well-grown algal strains are moved to a chlorophenicol (7.5 μgmL⁻¹)-containing liquid culture medium and cultured thereon.

The transgenic strains are cultured on an antibiotic-containing culture medium. A substantially complete loop of the transgenic strains or about 1.5 mL of microalgae is scratched and fetched. The microalgae are examined with a colonial polymerase chain reaction to determine whether the microalgae contain a BicA gene. Furthermore, the algal colonies are treated with TE-triton solution (TE, pH 8.0+1% Triton X-100) to achieve cellular suspension, and then the suspension is heated up at 95° C. for 3.5 min before being subjected to chloroform extraction twice. Then, the supernatant is fetched to undergo the polymerase chain reaction with the BicA primer pair. Eventually, the transgenic microalgae are examined to determine whether they contain any BicA gene. Upon completion of examination, whatever a BicA gene segment-containing transgenic microalga is regarded as a desirable transgenic strain.

4. Effect of CO₂ Concentration on Growth of Synechococcus sp. PCC7942 BicA Transgenic Strain and Photosynthesis Thereof

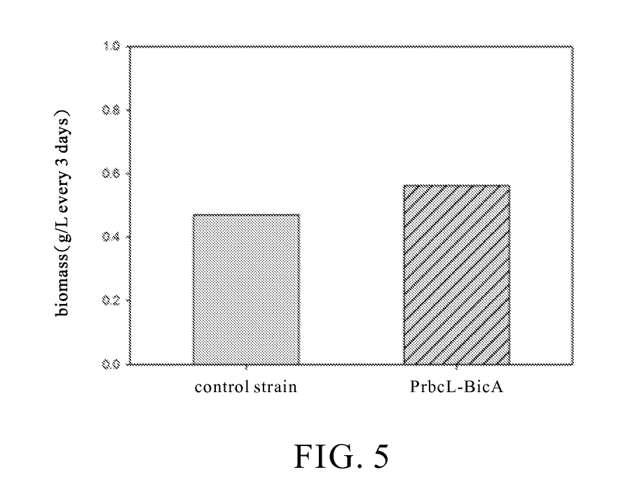

14 mL of a transgenic strain algal solution which has stayed still and been cultured for about five weeks is added to 500 ml of spectinomycin (2 μg/ml)-containing BG11+EPPS culture solution. Referring to FIG. 5, under irradiation of 150 E m⁻² s⁻¹ and 0.25 vvm of gas passing cultivation, the growth (OD₇₅₀) of a control strain and the BicA transgenic strain which are supplied with 2% CO₂/air is observed and measured. It is discovered that, in three days, when cultured with 2% CO₂/air, the yield of the biomass of the BicA transgenic strain increases to 0.56 g /L every three days. By contrast, the yield of the biomass of the control group equals 0.47 g/L every three days; hence, the yield of the biomass of the BicA transgenic strain is substantially 10% higher than that of the control group, indicating that the BicA transgenic strain grows faster than the control strain.

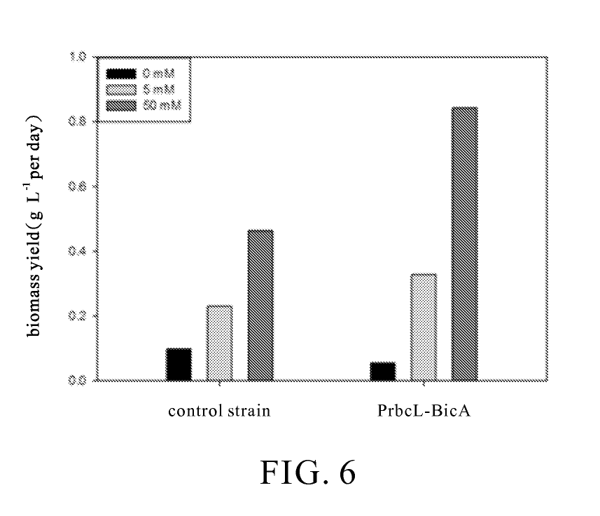

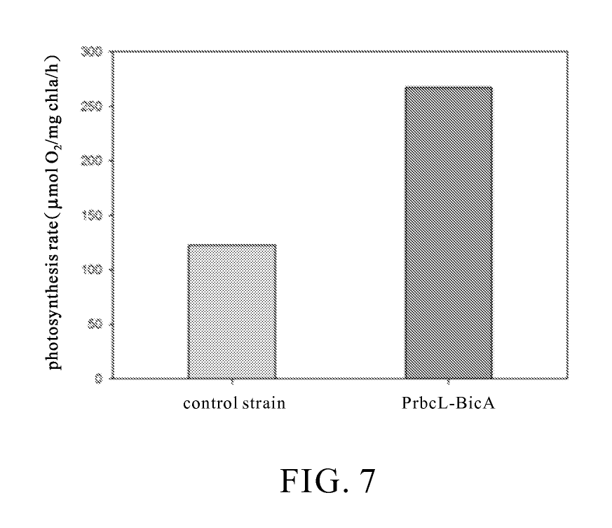

Under the irradiation of 150 E m⁻² s⁻¹, the growth (OD₇₅₀) of a control strain and the BicA transgenic strain which are supplied with NaHCO₃ of different concentration levels is observed and measured. It is discovered that, when cultured with 50 mM of NaHCO₃, the BicA transgenic strain grows faster than the control strain significantly. The yield of the biomass of the BicA transgenic strain equals 0.8430 g/L per day, which is 70% higher than that of the control group, that is, 0.550 g/L per day (see FIG. 6). Furthermore, the rate of photosynthesis performed by the BicA transgenic strain is twofold that of the control strain (see FIG. 7).

The result of the above experiments indicate that a method for enhancing cell growth of microalgae according to the present invention is effective in modifying microalgae genetically by gene transfer and enhancing transgenic expression of a bicarbonate transporter in microalgae, regardless of whether the bicarbonate transporter undergoes in-vivo cloning (as in embodiment 1) or in-vitro cloning (as in embodiment 2), and thus enhances the performance of the growth of the genetically modified microalgae, enhances the fixation of an inorganic carbon source of microalgae, and increases the photosynthesis rate and growth of the genetically modified microalgae, such that the genetically modified microalgae can be applied to the production of biofuels.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

What is claimed is:

1. A method for enhancing cell growth of microalgae by genetically modifying the microalgae by gene transfer, the method being characterized in that transgenic expression of a bicarbonate transporter (HCO₃⁻ transporter) in the microalgae is enhanced.

2. The method of claim 1, wherein a DNA sequence of the bicarbonate transporter is set forth by SEQ ID NO: 1.

3. The method of claim 1, wherein a DNA sequence of the bicarbonate transporter is set forth by SEQ ID NO: 2.

4. The method of claim 2, wherein a vector for enhancing transgenic expression of a bicarbonate transporter in microalgae is a transgenic vector pAM1573.

5. The method of claim 3, wherein a vector for enhancing transgenic expression of a bicarbonate transporter in microalgae is a transgenic vector pAM1573.

6. The method of claim 1, wherein the microalgae is one selected from the group consisting of Synechococcus, Thermosynechococcus, Cyanothece, Anabaena, Chlorella, and Chlamydomonas reinhardtii.


SEQUENCE LISTING (6 sequences)

SEQ ID NO: 1 - DNA, 1404 bp, Synechococcus PCC7942
atgactgtctggcaaactctgacttttgcccattaccaaccccaacagtggggccacagcagtttcttgcatcggctgtttggcagcctgcgagcttggcgggcctccagccagctgttggtttggtctgaggcactgggtggcttcttgcttgctgtcgtctacggttcggctccgtttgtgcccagttccgccctagggttggggctagccgcgatcgcggcctattgggccctgctctcgctgacagatatcgatctgcggcaagcaacccccattcactggctggtgctgctctactggggcgtcgatgccctagcaacgggactctcacccgtacgcgctgcagctttagttgggctagccaaactgacgctctacctgttggtttttgccctagcggctcgggttctccgcaatccccgtctgcgatcgctgctgttctcggtcgtcgtgatcacatcgctttttgtcagtgtctacggcctcaaccaatggatctacggcgttgaagagctggcgacttgggtggatcgcaactcggttgccgacttcacctcacgggtttacagctatctgggcaaccccaacctgctggctgcttatctggtgccgacgactgccttttctgcagcagcgatcggggtgtggcgcggctggctccccaagctgctggcgatcgctgcgacaggtgcgagcagcttatgtctgatcctcacctacagtcgcggtggctggctgggttttgtcgccatgatttttgtctgggcgttattagggctctactggtttcaaccccgtctacccgcaccctggcgacgctggctattcccagtcgtattgggtggactagtcgcggtgctcttggtggcggtgcttggacttgagccgttgcgcgtgcgcgtgttgagcatctttgtggggcgtgaagacagcagcaacaacttccggatcaatgtctggctggcggtgctgcagatgattcaagatcggccttggctgggcatcggccccggcaataccgcctttaacctggtttatcccctctatcaacaggcgcgctttacggcgttgagcgcctactccgtcccgctggaagtcgcggttgagggcggactactgggcttgacggccttcgcttggctgctgctggtcacggcggtgacggcggtgcggcaggtgagccgactgcggcgcgatcgcaatccccaagccttttggttgatggctagcttggccggtttggcaggaatgctgggtcacggtctgtttgataccgtgctctatcgaccggaagccagtacgctctggtggctctgtattggagcgatcgcgagtttctggcagccccaaccttccaagcaactccctccagaagccgagcattcagacgaaaaaatgtag

SEQ ID NO: 2 - DNA, 1701 bp, Synechococcus PCC7002
atgcagataaccaacaaaattcactttaggaatatccgcggcgatatttttggcgggctaacggcggcggtcattgcgttgcccatggccctcgccttcggggtggcatccggtgccggggcagaagccggtctctggggtgctgtgcttgtgggcttctttgccgccctctttgggggaacccccaccctcatttccgaacccacagggccgatgacggtggttatgaccgctgtgattgcccatttcacggccagcgccgctactccagaagaaggtttggcgatcgcctttaccgtcgtgatgatggccggggtgttccaaattatttttggctccctcaaactcggcaaatacgtcaccatgatgccctacaccgtgatttctggcttcatgtcagggatcgggatcatcctggtcattttgcaattagcgcccttcctgggacaggcgagtccggggggcggcgtcatcggcacgctccaaaatttacccacactgctgagtaatattcaaccgggcgaaacagccttagctttaggcaccgtggcgatcatctggtttatgccagagaagtttaaaaaggtaatcccgccccaattggtcgcccttgtcttggggacagtaatcgccttttttgttttcccgccagaagtaagcgatctccgtcgcatcggtgaaattcgagctgggttcccagagctggtcagaccgagctttagtccggttgaattccagagaatgatcctcgatgcggcagtgctagggatgctcggttgtatcgatgccctcttgacgtctgtcgtcgccgatagcttgacccggacagagcataattccaacaaagaattaattggccaaggtctagggaacctcttttctggcttgttcggcgggattgctggggctggggccaccatggggactgtggtaaatatccagtccggtggtcgaacggcgctttctggattggtacgggcctttgtcctgttggttgtgatccttggggcggctagtttaacggcaaccatccccctggctgtgcttgctgggattgccttcaaggtcggggttgacatcattgactggagtttcctaaaacgggcccacgaaatttcccccaagggggcactgatcatgtacggcgtcattctgttgacggtcttagttgacttgattgtcgccgtgggtgtgggtgtgtttgtcgctaatgtgctcaccatcgaacggatgagtaatctccagtctgaaaaagtccaaacggttagtgatgctgacgataatatccgcctgactaccactgaaaaacgctggttggatgaaggccaaggccgtgttcttttgtttcaactcagtgggccgatgatctttggggtcgcaaaggcgatcgccagagaacacaatgcgatgggtgactgtgatgccctcgtctttgatatcggtgaagtgccccacatgggggttaccgcttccctagccttagaaaatgccattgaagaggccctcgacaaagaacgtcaggtttatattgtcggtgctgcgggccaaacccgtcgccgtctggaaaaactcaagctctttaagcgggttccccccgataaatgtttgatgtcgcgggaagaagccctcaagaatgccgtgctcggaatctatccccatttggcggatggtgttacggctcccagttcagagatgggttaa

SEQ ID NO: 3 - DNA, 26 bp, artificial
  ictB gene primer forward
aagaattcggatccatgactgtctgg

SEQ ID NO: 4 - DNA, 28 bp, artificial
  ictB gene primer reverse
aggaattcggtaccctacattttttcgt

SEQ ID NO: 5 - DNA, 55 bp, artificial
  bicA gene primer forward
aattcccgggtttaagaaggagatatacatatgcagataaccaacaaaattcact

SEQ ID NO: 6 - DNA, 31 bp, artificial
  bicA gene primer reverse
aattcccgggttaacccatctctgaactggg